(19) United States
(12) Patent Application Publication
(10) Pub. No.: US 2016/0096788 A1
(43) Pub. Date: Apr. 7, 2016

(54) LIQUID CRYSTAL COMPOSITION HAVING A NEGATIVE LIQUID CRYSTAL MODE

(71) Applicant: SAMSUG DISPLAY CO., LTD., Yongin-City (KR)

(72) Inventors: Ji-Hong BAE, Gyeonggi-do (KR); Kang-Seob JEONG, Gyeonggi-do (KR); Mi-Suk KIM, Gyeonggi-do (KR); Keun-Chan OH, Gyeonggi-do (KR); Tae-Ho KIM, Gyeonggi-do (KR); Beom-Soo SHIN, Gyeonggi-do (KR); Kyung-Hee LEE, Gyeonggi-do (KR); Kyung-Sean TAK, Gyeonggi-do (KR)

(21) Appl. No.: 14/748,901

(22) Filed: Jun. 24, 2015

(30) Foreign Application Priority Data
Oct. 7, 2014 (KR) 10-2014-0135179

(51) Int. Cl. C07C 25/18; C09K 19/30

(52) U.S. Cl. CPC C07C 25/18; C09K 19/3003; C09K 2019/3004; C09K 2019/3009; C09K 2019/301; C09K 2019/3016

(57) ABSTRACT

Provided is a liquid crystal composition including an additive of the following chemical formula:

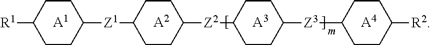

### CROSS-REFERENCE TO RELATED APPLICATION

This application claims priority under 35 U.S.C. §119 to Korean Patent Application No. 10-2014-0135179, filed on Oct. 7, 2014, in the Korean Intellectual Property Office, the disclosure of which is incorporated by reference herein in its entirety.

### TECHNICAL FIELD

Exemplary embodiments of the present invention relate to a liquid crystal composition, and more particularly to a liquid crystal composition having a negative liquid crystal mode.

### DISCUSSION OF RELATED ART

In liquid crystal displays, a μ-slit pattern may be used to overcome limitations of transmissivity in a Patterned Vertical Alignment (PVA) mode. A Super Vertical Alignment (SVA) mode may be used when a stable orientation of liquid crystals is induced through a polymerization reaction by using a Reactive Mesogen (RM) and a UV process in order to produce texture stability and pretilt angle of liquid crystals. In a 3D phase, high speed response display, a low viscosity alkenyl compound may be used. Therefore, there may be a need for a reliable alkenyl compound for use in an LCD manufacturing process such as RM photocuring.

Negative Plane Line Switching (PLS) Is being developed to overcome the limitations of transmissivity in the existing positive Plane Line Switching (PLS). Negative PLS may be used in a liquid crystal display apparatus including liquid crystals having a negative dielectric permittivity.

In a negative PLS display, the liquid crystals may be rubbed in a direction vertical to an electrode, and transmissivities of an electrode center and a slit center may be higher than that of a positive PLS.

Therefore, the development of negative liquid crystals may be focused on a stabilizer for reducing ion impurities inside a liquid crystal mixture.

A low viscosity liquid crystal may be used, and a neutral alkenyl may be used. However, as an alkenyl compound is used, a radical source may be generated in an orientation film. The radical source may react with the alkenyl compound, generating a polar substance which may react with surrounding ions, thereby causing a problem of Voltage Holding Ratio (VHR) drop.

In a SVA phase liquid crystal display containing an alkenyl compound, alkenyl byproducts such as an aldehyde may be produced after an electric field exposure process and fluorescent exposure process and a linear afterimage may occur.

Afterimages may occur in negative PLS liquid crystal displays, and adding a stabilizer (e.g., HALS type) may reduce the occurrence of a linear afterimage.

### SUMMARY

Exemplary embodiments of the present invention provide a liquid crystal composition having a highly reliable negative liquid crystal mode.

According to an exemplary embodiment of the present invention there is provided a liquid crystal composition including an additive of chemical formula 1:

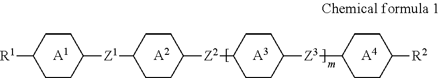

Each of R¹ and R² may independently represent an alkyl having 1 to 12 carbon atoms, a hydrogen, a halogen or a cyano, respectively, and one or two nonadjacent CH₂ groups may be replaced by —O—, —CH═CH—, —CO—, —OCO— or —COO— such that an oxygen atom is not directly connected to another oxygen atom.

A¹, A², A³, and A⁴ may each independently be:

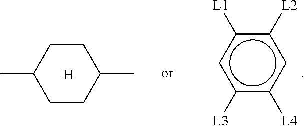

One, two, or three radicals of A¹, A², A³ and A⁴ may independently be:

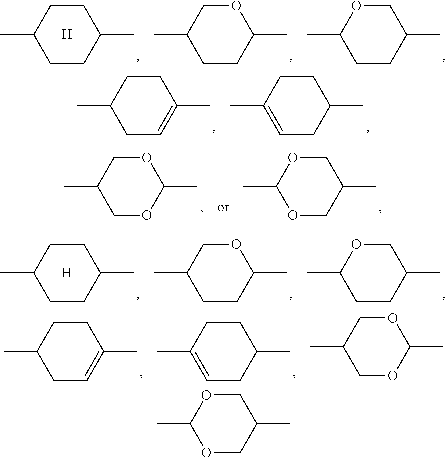

L1 to L4 may each independently be —H, —F, —Cl, —OCF₃, —CF₃, CH₂F, or CHF₂.

Each of Z¹, Z² and Z³ may independently be —COO—, —OCO—, —CF₂O—, —OCF₂—, —CH₂O, —OCH₂—, —SCH₂—, —CH₂S—, —CH₂CH₂—, —C₂F₄—, —CH₂—CF₂—, —CF₂CH₂—, —(CH₂)z-, —CH═CH—, —CF═CF—, —CH═CF—, —CF═CH—, —C═C—, —CH═CHCH₂O—, or a single bond. When m=0, Z¹ and Z² do not represent a single bond at the same time, z is 3, 4, 5, or 6, and m is 0 or 1.

The additive of chemical formula 1 in the composition may be 0.001 wt % to 0.35 wt % based on a total weight of the composition.

The liquid crystal composition may include an alkenyl compound, an alkoxy compound, a hydroquinone compound, a terphenyl compound, or a tolane compound.

The alkenyl compound may be at least one selected from:

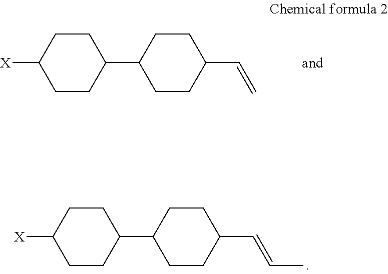

Each X may independently be an alkyl group having 1 to 5 carbon atoms.

The alkoxy compound may be at least one selected from:

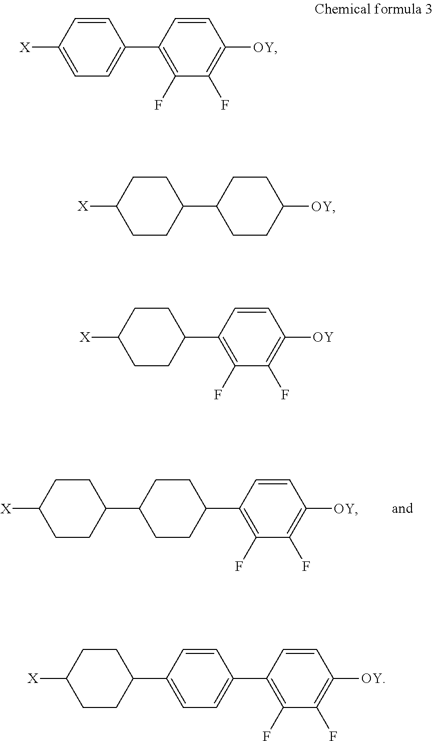

Each of X and Y may be an alkyl group having 1 to 5 carbon atoms.

The terphenyl compound may have a structure as below:

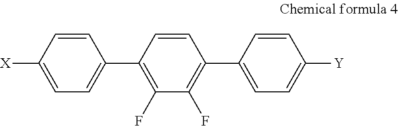

Each of X and Y may be an alkyl group having 1 to 5 carbon atoms.

The liquid crystal composition need not include reactive mesogen (RM).

The liquid crystal composition may include one or more types of reactive mesogen (RM).

The liquid crystal composition may include a BHT (butylated hydroxytoluene) stabilizer or HALS (hindered amine light stabilizer), or an optical initiator.

The stabilizer may alkylate monophenol, alkylthiomethylphenol, hydroquinone and alkylate hydroquinone, tocopherol, hydroxylate thiodiphenyl ether, alkylidenebisphenol, O-, N- and S-benzyl compound, hydroxybenzylate malonate, aromatic hydroxybenzyl compound, benzylphosphonate, acylaminophenol, monovalent or multivalent alcohol, monovalent or multivalent alcohol and β-(3,5-di-tertbutyl-4-hydroxyphenyl)-propionic acid ester, monovalent or multivalent alcohol and 13-(5-tertbutyl-4-hydroxy-3-methylphenyl)propionic acid ester, monovalent or multivalent alcohol and β-(3,5-dicyclohexyl-4-hydroxyphenyl)-propionic acid ester, monovalent or multivalent alcohol and 3,5-di-tertbutyl-4-hydroxyphenyl acetic acid ester, β-(3,5-di-tertbutyl-4-hydroxyphenyl)propionic acid amide, ascorbic acid, and amine antioxidant, or a hindered amine compound.

The liquid crystal composition may be used in a liquid crystal device including a general VA orientation film, a PLS orientation film (rubbing orientation film), or an orientation film including a reactive mesogen (RM), or no orientation film.

The liquid crystal composition according to exemplary embodiments of the present invention may reduce the occurrence of panel afterimage and increase reliability VHR drop by using a 4-ring compound as an additive.

### BRIEF DESCRIPTION OF THE DRAWINGS

The above and other features of the present invention will become more apparent by describing in detail exemplary embodiments thereof with reference to the accompanying drawings, in which:

FIG. 1 is a graph illustrating a voltage holding ratio (VHR) of a liquid crystal composition according to an exemplary embodiment of the present invention and a reference example; and

FIGS. 2 to 4 illustrate changes in optical characteristics according to an exemplary embodiment of the present invention and a reference example.

### DETAILED DESCRIPTION

Hereinafter, exemplary embodiments of the present invention will be described in more detail with reference to the accompanying drawings. Exemplary embodiments of the present invention may be described herein with reference to cross-sectional illustrations that are schematic illustrations of exemplary embodiments. Variations from the shapes of the Illustrations as a result, for example, of manufacturing techniques and/or tolerances, may occur. Thus, exemplary embodiments of the present invention should not be construed as limited to the particular shapes of regions illustrated herein but may include deviations in shapes that result, for example, from manufacturing. In the specification and drawings, lengths and sizes of layers and regions may be exaggerated for clarity. Like reference numerals may refer to like elements throughout the specification and drawings.

Terms such as ‘first’ and ‘second’ may be used to describe various components, but they should not limit the various components.

A liquid crystal composition according to an exemplary embodiment of the present invention may include a 4-ring compound having the following chemical formula I. The liquid crystal composition of formula I may reduce a generation of ion impurities in a liquid crystal

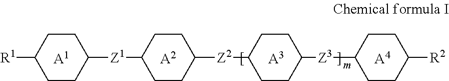

Each of R¹ and R² may be an alkyl having 1 to 12 carbon atoms, hydrogen, a halogen, or a cyano. One or two nonadjacent —CH₂— groups of R¹ or R² may be replaced by —O—, —CH═CH—, —CO—, —OCO— or —OO— such that an oxygen atom is not directly connected to another oxygen atom.

Each of A¹, A², A³, and A⁴ may be one of the following structures:

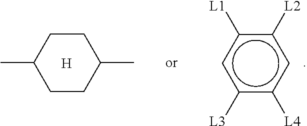

One, two, or three radicals of A¹, A², A³ and A⁴ may each be one of the following structures:

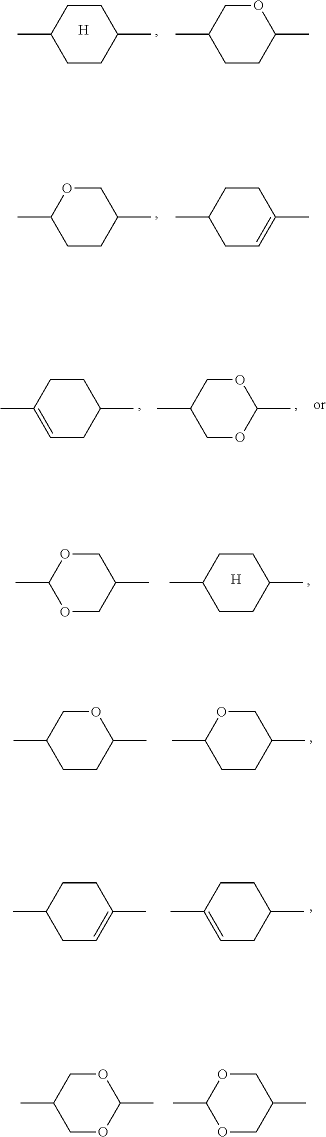

Each of L1 to L4 may be H, F, Cl, OCF₃, CF₃, CH₂F, or CHF₂.

Each of Z¹, Z², and Z³ may be —COO—, —OCO—, —CF₂O—, —OCF₂—, —CH₂O, —OCH₂—, —SCH₂—, —CH₂S—, —CH₂CH₂—, —C₂F₄—, —CH₂—CF₂—, —CF₂CH₂—, —(CH₂)z-, —CH═CH—, —CF═CF—, —CH═CF—, —CF═CH—, —C═C—, or —CH═CHCH₂O—, or a single bond. When m=O, Z¹ and Z² do not represent a single bond at the same time, z may be 3, 4, 5, or 6, and m is 0 to 1.

For a unit compound that forms a liquid crystal having a negative (−) dielectric anisotropy (Δ∈), a negative (−) polar compound may be used to generate polarity characteristics, and a neutral compound may be used for reducing viscosity. When a pretilt angle of a liquid crystal is formed by using RM (Reactive Mesogen), such as a Super Vertical Alignment (SVA), a terphenyl compound having a relatively long UV energy absorption wavelength range may be used in order to increase the RM reactivity in a UV photocuring process. The terphenyl compound may transfer UV energy and may increase the RM reactivity. However, the terphenyl compound may also transfer too much UV energy, which may result in damage to the liquid crystal mixture itself and may reduce the Voltage Holding Ratio (VHR) of the liquid crystal mixture.

The 4-ring compound may absorb external energy such as light/heat/initiator and may reduce an unstable free radical reaction, thereby having a function as a radical scavenger.

The 4-ring compound may have a composition ratio of 0.001 wt % to 0.35 wt % based on a total weight of the liquid crystal composition. When the 4-ring compound has a composition ratio of less than 0.001 wt %, it may have a relatively small effect, whereas when the 4-ring compound has a composition ratio exceeding 0.35 wt %, it may have a greater effect than is desired.

The liquid crystal composition according to exemplary embodiments of the present invention may be used in a liquid crystal display apparatus. For example, the liquid crystal composition may be used in a liquid crystal display apparatus having any structure including an upper and lower panel which both have a transparent electrode, or a substrate having an electrode capable of applying one or more voltages, or a structure in which a fringe field area of a vertical or horizontal direction may exist inside the liquid crystal display apparatus.

The liquid crystal composition according to exemplary embodiments of the present invention may be used in any method using an electric field and a magnetic field capable of driving liquid crystals.

The 4-ring compound included in the liquid crystal composition according to exemplary embodiments of the present invention may be used as an additive and may be used together with a compound such as an alkenyl compound, an alkoxy compound, a terphenyl compound, a hydroquinone compound, or a tolane compound. The compound (used together with the 4-ring compound) may have a composition ratio of 99.65 wt % to 99.999 wt % based on the total weight of the liquid crystal composition.

The alkenyl compound may be a low viscosity neutral compound. Since the alkenyl compound has a relatively lower viscosity than other compounds, it may have a relatively high response speed. The alkenyl compound may be at least one selected from a liquid crystal represented by the following chemical formula 2.

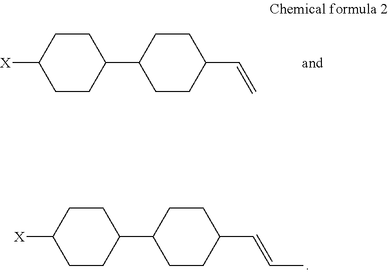

X may be an alkyl radical having 1 to 5 carbon atoms.

The alkoxy compound may be a low polar neutral compound. Since the alkoxy compound has a relatively lower viscosity than other compounds, it may have a relatively high response speed. The alkoxy compound may be at least one selected from a liquid crystal represented by the following chemical formula 3.

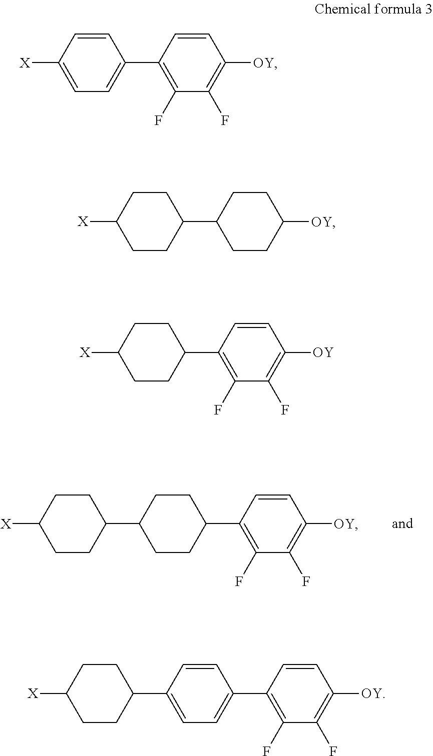

Each of X and Y may be an alkyl group having 1 to 5 carbon atoms.

The terpenyl compound may be represented by the following chemical formula 4.

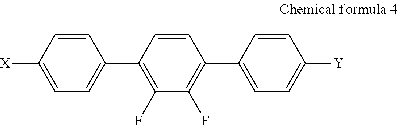

Each of X and Y may be an alkyl group having 1 to 5 carbon atoms.

The liquid crystal composition according to exemplary embodiments of the present invention may include RM. The liquid crystal composition may include one or more types of RM.

In the liquid crystal composition according to exemplary embodiments of the present invention, a BHT type stabilizer or HALS type stabilizer may be included separately or in combination with the liquid crystal composition. The BHT type stabilizer or HALS type stabilizer may be within a range of about 0.001 wt % to about 5 wt % of the liquid crystal composition.

The BHT type stabilizer may be at least one selected from alkylate monophenol, alkylthiomethylphenol, hydroquinone and alkylate hydroquinone, tocopherol, hydroxylate thiodiphenyl ether, alkylidenebisphenol, O-, N- and S-benzyl compound, hydroxybenzylate malonate, aromatic hydroxybenzyl compound, benzylphosphonate, acylaminophenol, monovalent or multivalent alcohol, monovalent or multivalent alcohol and β-(3,5-di-tertbutyl-4-hydroxyphenyl)-propionic acid ester, monovalent or multivalent alcohol and 3-(5-tertbutyl-4-hydroxy-3-methylphenyl)-propionic acid ester, monovalent or multivalent alcohol and β-(3,5-dicyclohexyl-4-hydroxyphenyl)propionic acid ester, monovalent or multivalent alcohol and 3,5-di-tertbutyl-4-hydroxyphenyl acetic acid ester, β-(3,5-di-tertbutyl-4-hydroxyphenyl)propionic acid amide, ascorbic acid, and amine antioxidant.

A hindered amine compound may be used as the HALS type stabilizer. For example, a 2,2,6,6-tetraalkylpiperidine derivative may be used as the HALS type stabilizer.

The stabilizer may be, but is not limited to, the following stabilizers.

(1) Alkylate monophenol, for example, 2,6-di-tertbutyl-4-methylphenol, 2-tertbutyl-4,6-dimethylphenol, 2,6-di-tertbutyl-4-ethylphenol, 2,6-di-tertbutyl-4-n-butyphenol, 2,6-di-tertbutyl-4-isobutylphenol, 2,6-di-cyclopentyl-4-methylphenol, 2-(α-methylcyclohexyl)-4,6-dimethylphenol, 2,6-di-octadecyl-4-methylphenol, 2,4,6-tricyclohexylphenol, or 2,6-di-tertbutyl-4-methoxymethylphenol. Nonylphenol branched from a straight chain or side chain, for example, 2,6-di-nonyl-4-methylphenol, 2,4-dimethyl-6-(1′-methyl-undec-1′-yl)-phenol, 2,4-dimethyl-6-(1′-methyl-heptadec-1′-yl)-phenol, 2,4-dimethyl-6-(1′-methyltridec-11-yl)-phenol, or a compound thereof.

(2) Alkylthiomethylphenol, for example, 2,4-di-octylthiomethyl-6-tertbutylphenol, 2,4-di-octylthiomethyl-6-methylphenol, 2,4-dioctylthiomethyl-6-ethylphenol, or 2,6-di-dodecylthiomethyl-4-nonylphenol.

(3) Hydroquinone and alkylate hydroquinone, for example, 2,6-di-tertbutyl-4-methoxyphenol, 2,5-di-tertbutyl-hydroquinone, 2,5-di-tertamylhydroquinone, 3,6-diphenyl-4-octadecyloxyphenol, 2,6-di-tertbutyl-hydroquinone, 2,5-di-tertbutyl-4-hydroxyanisol, 3,5-di-tertbutyl-4-hydroxyanisol, 3,5-di-tertbutyl-4-hydroxyphenyl stearate, or bis(3,5-di-tertbutyl-4-hydroxyphenyl)adiphate.

(4) Tocopherol, for example, α-tocopherol, β-tocopherol, γ-tocopherol, δ-tocopherol or a compound thereof (e.g., Vitamin E).

(5) hydroxylate thiodiphenyl ether, for example, 2,2′-thiobis(6-tertbutyl-4-methylphenol), 2,2′-thiobis(4-octylphenol), 4,4′-thiobis(6-tertbutyl-3-methylphenol), 4,4′-thiobis(6-tertbutyl-2-methylphenol), 4,4′-thiobis(3,6-di-secamylphenol), or 4,4′-bis(2,6-dimethyl-4-hydroxyphenyl)disulflde.

(6) Alkylidenebisphenol, for example, 2,2′-methylenebis(6-tertbutyl-4-methylphenol), 2,2′-methylenebis(6-tertbutyl-4-ethylphenol), 2,2′-methylenebis[4-methyl-6-(α-methylcyclohexyl)-phenol], 2,′2-methylenebis(4-methyl-6-cyclohexylphenol),2,2′-methylenebis(6-nonyl-4-methylphenol), 2,2′-methylenebis(4,6-di-tertbutylphenol), 2,2′-ethylidenebis(4,6-di-tertbutylphenol), 2,2′-ethylidenebis(6-tertbutyl-4-isobutylphenol), 2,2′-methylenebis[6-(α-methylbenzyl)-4-nonylphenol], 2,2′-methylenebis[6-(α,α-dimethylbenzyl-4-nonylphenol), 4,4′-methylenebis(2,6-di-tertbutylphenol), 4,4′-methylenebis[6-tertbutyl-2-methylphenol], 1,1-bis(5-tertbutyl-4-hydroxy-2-methylphenyl)butane, 2,6-bis(3-tertbutyl-5-methyl-2-hydroxybenzyl)-4-methylphenol, 1,1,3-tris(5-tertbutyl-4-hydroxy-2-methylphenyl)butane, 1,1-bis(5-tertbutyl-4-hydroxy-2-methyl-phenyl)-3-n-dodecylmercaptobutane, ethylene glycolbis[3,3-bis(3′-tertbutyl-4′-hydroxyphenyl)butyrate], bis(3-tertbutyl-4-hydroxy-5-methyl-phenyl)dicyclopentadiene, bis[2-(3′-tertbutyl-2′-hydroxy-5′-methylbenzyl)-6-tertbutyl-4-methylphenyl]terephthalate, 1,1-bis-(3,5-dimethyl-2-hydroxyphenyl)butane, 2,2-bis(3,5-di-tertbutyl-4-hydroxyphenyl)-propane, 2,2-bis(5-tertbutyl-4-hydroxy-2-methylphenyl)-4-n-dodecylmercaptobutane, or 1,1,5,5-tetra(5-tertbutyl-4-hydroxy-2-methylphenyl)pentane.

(7) An O-, N- and S-benzyl compound for example, 3,5,3′,5′-tetra-tertbutyl-4,4′-dihydroxy-dibenzyl ether, octadecyl-4-hydroxy-3,5-dimethylbenzylmercaptoacetate, tridecyl-4-hydroxy-3,5-di-tertbutylbenzylmercaptoacetate, tris(3,5-di-tertbutyl-4-hydroxybenzyl)amine, bis(4-tertbutyl-3-hydroxy-2,6-dimethylbenzyl)dithioterephthalate, bis(3,5-di-tertbutyl-4-hydroxybenzyl)sulfide, or isooctyl-3,5-di-tertbutyl-4-hydroxybenzyl mercaptoacetate.

(8) Hydroxybenzylate malonate, for example, dioctadecyl-2,2-bis(3,5-di-tertbutyl-2-hydroxybenzyl)malonate, di-octadecyl-2-(3-tertbutyl-4-hydroxy-5-methylbenzyl)-malonate, di-dodecylmercaptoethyl-2,2-bis(3,5-di-tertbutyl-4-hydroxybenzyl)malonate, or bis-[4-(1,1,3,3-tetramethylbutyl)phenyl]-2,2-bix(3,5-di-tertbutyl-4-hydroxybenzyl)malonate.

(9) Aromatic hydroxybenzyl compound, for example, 1,3,5-tris(3,5-di-tertbutyl-4-hydroxybenzyl-2,4,6-trimethylbenzene, 1,4-bis(3,5-di-tertbutyl-4-hydroxybenzyl)-2,3,5,6-tetramethylbenzene, or 2,4,6-tris(3,5-di-tertbutyl-4-hydroxybenzyl)phenol.

(10) Triazine compound, for example, 2,4-bis(octylmercapto)-6-(3,5-di-tertbutyl-4-hydroxyanilino)-1,3,5-triazine, 2-octylmercapto-4,6-bis(3,5-di-tertbutyl-4-hydroxyanilino)-1,3,5-triazine, 2-octylmercapto-4,6-bis(3,5-di-tertbutyl-4-hydroxyphenoxy)-1,3,5-triazine, 2,4,6-tris(3,5-di-tertbutyl-4-hydroxyphenoxy)-1,2,3-triazine, 1,3,5-tris(3,5-di-tertbutyl-4-hydroxybenzyl)isocyanurate, 1,3,5-tris(4-tertbutyl-3-hydroxy-2,6-dimethylbenzyl)isocyanurate, 2,4,6-tris(3,5-di-tertbutyl-4-hydroxyphenylethyl)-1,3,5-triazine, 1,3,5-tris(3,5-di-tertbutyl-4-hydroxyphenylproprionyl)hexahydro-1,3,5-triazine, or 1,3,5-tris(3,5-dicyclohexyl-4-hydroxybenzyl)isocyanurate.

(11) Benzyl phosphonate, for example, dimethyl-2,5-di-tertbutyl-4-hydroxybenzyl phosphonate, diethyl-3,5-di-tertbutyl-4-hydroxybenzylphosphonate, dioctadecyl-3,5-di-tertbutyl-4-hydroxybenzylphosphonate, dioctadecyl-5-tertbutyl-4-hydroxy-3-methylbenzylphosphonate, or 3,5-di-tertbutyl-4-hydroxybenzyl-phosphonic acid monoethyl ester calcium salt.

(12) Acylaminophenol, for example, 4-hydroxyrauranilide, 4-hydroxystearanilide, or octyl N-(3,5-di-tertbutyl-4-hydroxyphenyl)carbamate.

(13) Monovalent or multivalent alcohol and 6-(3,5-di-tertbutyl-4-hydroxyphenyl)-propionic acid ester. The monovalent or multivalent alcohol may be, for example, methanol, ethanol, n-octanol, i-octanol, octadecanol, 1,6-hexandiol, 1,9-nonadiol, ethylene glycol, 1,2-propandiol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiopentadecanole, trimethylhexandiole, trimethylolpropane, or 4-hydroxymethyl-1-phospa-2,6,7-trioxabicyclo[2.2.2]octane.

(14) Monovalent or multivalent alcohol and 6-(5-tertbutyl-4-hydroxy-3-methylphenyl)-propionic acid ester. The monovalent or multivalent alcohol may be, for example, methanol, ethanol, n-octanol, i-octanol, octadecanol, 1,6-hexandiol, 1,9-nonanediol, ethylene glycol, 1,2-propandiol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexandiol, trimethylolpropane, 4-hydroxymethyl-1-phospa-2,6,7-trioxabicyclo-[2.2.2]octane, or 3,9-bis[2-{3-(3-tertbutyl-4-hydroxy-5-methylphenyl)prophionyloxy}-1,1-dimethylethyl]-2,4,8,10-tetraoxaspiro[5.5]-undecane.

(15) Monovalent or multivalent alcohol and β-(3,5-dicyclohexyl-4-hydroxyphenyl)-propionic acid ester. The monovalent or multivalent alcohol may be, for example, methanol, ethanol, octanol, octadecanol, 1,6-hexandiol, 1,9-nonanediol, ethylene glycol, 1,2-propandiol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexandiol, trimethylpropane, or 4-hydroxymethyl-1-phospa-2,6,7-trioxabicyclo-[2.2.2]octane.

(16) Monovalent or multivalent alcohol and 3,5-di-tertbutyl-4-hydroxyphenyl acetic acid ester. The monovalent or multivalent alcohol may be, for example, methanol, ethanol, octanol, octadecanol, 1,6-hexandiol, 1,9-nonanediol, ethylene glycol, 1,2-propandiol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexandiol, trimethylolpropane, or 4-hydroxymethyl-1-phospa-2,6,7-trioxabicyclo-[2.2.2]octane.

(17) 6-(3,5-di-tertbutyl-4-hydroxyphenyl)propionic acid amide, for example, N,N′-bis(3,5-di-tertbutyl-4-hydroxyphenylpropionic acid)hexamethylenediamide, N,N′-bis(3,5-di-tertbutyl-4-hydroxyphenylpropionyl)trimethylenediamide, N,N′-bis(3,5-di-tertbutyl-4-hydroxyphenylpropionyl)hydrazide, or N,N′-bis[2-3-[3,5-di-tertbutyl-4-hydroxyphenyl]propionyloxy)ethyl]oxamide.

(18) Ascorbic acid (e.g., Vitamin C).

(19) An Amine antioxidant, for example, N,N′-di-isopropyl-p-phenylenediamine, N,N′-di-secbutyl-p-phenylenediamine, N,N′-bis(1,4-dimethylpentyl)-p-phenylenediamine, N,N′-bis(1-ethyl-3-methylpentyl)-p-phenylenediamine, N,N′-bis(l-methylheptyl)-p-phenylenediamine, N,N′-dicyclohexyl-p-phenylenediamine, N,N′-diphenyl-p-phenylenediamine, N,N′-bis(2-napthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)-diphenylamine, N,N′-dimethyl-N,N′-di-secbutyl-p-phenylenediamine, diphenylamine, N-alyldiphenylamine, 4-Isopropoxydiphenylamine, N-phenyl-1-napthylamine, N-(4-tertoctylphenyl)-1-napthylamine, N-phenyl-2-napthylamine, octylate diphenylamine, for example, p,p′-di-tertoctyldiphenylamine, 4-n-butylaminophenol, 4-butyrylaminophenol, 4-nonaneoylaminophenol, 4-dodecanoylaminophenol, 4-octadecanoylaminophenol, bis(4-methoxyphenyl)amine, 2,6-di-tertbutyl-4-dimethylaminomethylphenol, 2,4′-di-aminodiphenylmethane, 4,4′-diaminodiphenylmethane, N,N,N′,N′-tetramethyl-4,4′-di-aminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tertoctylate N-phenyl-1-naphthylamine, mono- and dialkylate tertbutyl/tertoctyldiphenylamine compound, mono- and dialkylate nonyldiphenylamine compound, mono- and dialkylate dodecyldiphenylamine compound, mono- and dialkylate isopropyl/isohexyldiphenylamine compound, mono- and dialkylate tertbutyldiphenylamine compound, 2,3-di-hydro-3,3-dimethyl-4H-1,4-benzotiazine, phenothiazine, mono and dialkylate tertbutyl/tertoctylphenothiazine compound, mono- and dialkylate tertoctyl-phenothiazine compound, N-alylphenothiazine, N,N,N′,N′-tetraphenyl-1,4-diaminobut-2-ene, N,N-bis(2,2,6,6-tetramethylpiperide-4-yl-hexamethylenediamine, bis(2,2,6,6-tetramethylpiperide-4-yl)sebacate, 2,2,6,6-tetramethylpiperidine-4-one, or 2,2,6,6-tetramethylpiperidine-4-ole.

The liquid crystal composition according to an exemplary embodiment of the present invention may include a photoinitator that may be 0.001 wt % to 1 wt % of the liquid crystal composition. The photoinitiator may be a general photoinitiator known in the field. For example, the photoinitiator may be 1-hydroxy-cyclohexyl-phenyl-ketone (IRGACURE® 184), IRGACURE® 184 (50 wt %)+benzophenone (50 wt %) (IRGACURE® 500), 2-hydroxy-2-methyl-phenyl-1-propanone (DAROCUR® 1173), 2-hydroxy-1-[4-2(-hydroxyethoxy)phenyl]-2-methyl-1-propane (IRGACURE® 2959), methylbenzoylformate (DAROCUR® MBF), oxy-phenyl-acetic acid 2-[2-oxy-2-phenyl-acetoxy-ethoxy-ethyl ester and oxy-phenyl-acetic 2-[hydroxyl-ethosy]-ethyl ester (IRGACURE® 754), alpha, alpha-dimethoxy-alpha-phenylacetophenone (IRGACURE® 651), or 2-benzyl-2-(dimethylamino)-1-[4-(4-morphorynol)phenyl]-1-butanone (IRGACURE® 369), 2-methyl-1-[4-(methylthio)phenyl]-2-(4-morphorynyl)-1-propanone (IRGACURE® 907), IRGACURE® 369 (30 wt %)+IRGACURE® 651 (70 wt %)(IRGACURE® 1300), diphenyl(2,4,6-trimethylbenzoyl)-phosphine oxide (DUROCUR® TPO), DUROCUR® TPO (50 wt %)+DAROCUR® 1173 (50 wt %)(DAROCUR® 4265), phosphine oxide, phenyl bis(2,4,6-trimethyl benzoyl)(IRGACURE® 819), (IRGACURE® 819 (45% activity) (IRGACURE® 819DW), or IRGACURE® 819 (20 wt %)+DAROCUR® 1173 (80 wt %)(IRGACURE® 2022) dispersed in water and phosphine oxide may be used.

The liquid crystal composition according to an exemplary embodiment of the present invention may be used in a liquid crystal device having a general vertical alignment (VA) orientation film, a PLS orientation film (e.g., a rubbing orientation film), an orientation film including RM (e.g., a surface-stabilized vertical alignment (SS-VA) display), or in a liquid crystal device having no orientation film. The liquid crystal composition according to an exemplary embodiment of the present invention may be used in an optical orientation film.

### Embodiment 1

A liquid crystal composition according to an exemplary embodiment of the present invention may have the following structure including 0.3 wt % of a 4-ring compound.

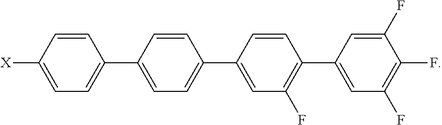

X may be an alkyl group having 3 carbon atoms.

### Reference Example

A liquid crystal composition according to a reference example may have the composition as in table 1 below.

TABLE 1

| | | Reference | |
|---|---|---|---|
| | | Example | Embodiment 1 |
| 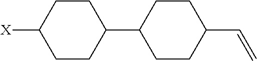 | 3 | 15% | 14.96% |
| 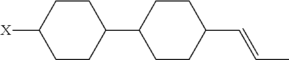 | 3 | 9% | 8.97% |
| 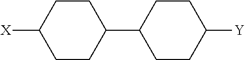 | 2, 3 3, 4 | 8% 7.5% | 7.98% 7.48% |
| 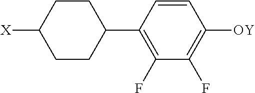 | 3, 4 | 7% | 6.98% |
| 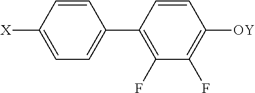 | 3, 2 | 16% | 15.95% |
| 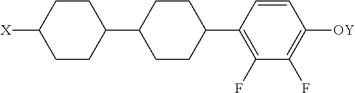 | 3, 2 5, 2 | 10% 8% | 9.97% 7.98% |
| 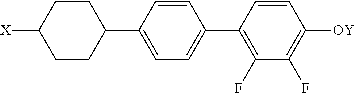 | 2, 2 3, 2 | 3% 8.5% | 2.99% 8.47% |
| 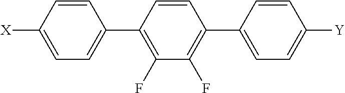 | 2, 3 | 8% | 7.97% |
| 4-ring single | | | 0.30% |

In the above table 1, the numbers on the right side of the compositions represent the number of carbon atoms for X and Y, respectively. X and Y may represent alkyl groups.

### Example 1

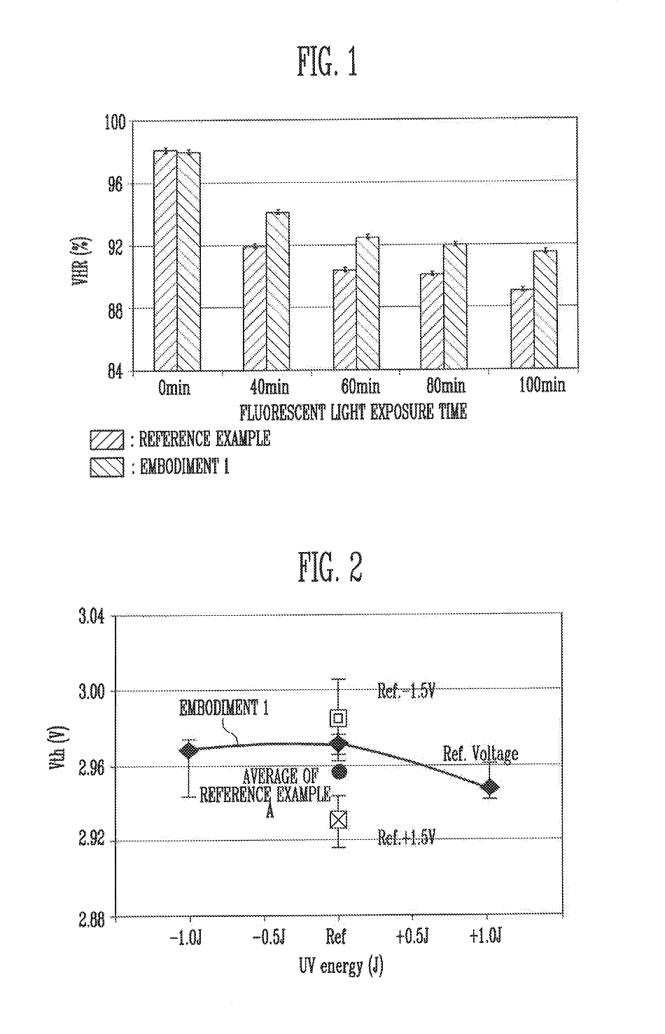

Voltage Holding Ratios (VHRs) for the liquid crystal composition of embodiment 1 and the reference example are illustrated in table 2 below and FIG. 1. VHRs may be measured by radiating the liquid crystal composition with light (e.g., after 0 minutes, 40 minutes, 60 minutes, 80 minutes, and 100 minutes of fluorescent light exposure).

TABLE 2

| | Reference example | | | | | Embodiment 1 | | | | |
|---|---|---|---|---|---|---|---|---|---|---|
| | (min) | | | | | | | | | |
| | 0 | 40 | 60 | 80 | 100 | 0 | 40 | 60 | 80 | 100 |
| S/S | 6 | 6 | 6 | 6 | 6 | 6 | 6 | 6 | 6 | 6 |
| Min | 97.89 | 91.78 | 90.14 | 89.93 | 88.96 | 97.85 | 93.92 | 92.36 | 91.68 | 91.30 |
| Max | 98.19 | 92.13 | 90.57 | 90.23 | 89.16 | 98.16 | 94.21 | 92.66 | 92.24 | 91.68 |
| AVG | 98.06 | 91.93 | 90.37 | 90.06 | 89.05 | 97.98 | 94.11 | 92.45 | 91.94 | 91.48 |
| Stdev | 0.15 | 0.15 | 0.18 | 0.13 | 0.09 | 0.15 | 0.13 | 0.14 | 0.23 | 0.16 |

As can be seen from table 2 and FIG. 1, the liquid crystal composition of embodiment 1 has increased VHR compared with the liquid crystal composition of the reference example.

### Example 2

Physical characteristics of the liquid crystal composition according to embodiment 1 and the reference example may be compared for each case where the content of the 4-ring compound is 0.1 wt %, 0.2 wt %, and 0.3 wt %. Table 3 below illustrates physical characteristics of the liquid crystal composition according to embodiment 1 and the reference example, and then a marginal content where there are no changes in optical characteristics may be selected.

TABLE 3

| | Reference | 4-ring compound content (wt %) | | |
|---|---|---|---|---|
| | example | 0.1 wt % | 0.2 wt % | 0.3 wt % |
| Tni | 100.0% | 100.0% | 100.3% | 100.4% |
| Δn | 100.0% | 100.0% | 100.3% | 100.5% |
| Δε | 100.0% | 99.4% | 99.1% | 98.4% |
| γ1 | 100.0% | 100.0% | 101.0% | 101.0% |
| K33 | 100.0% | 99.4% | 99.4% | 99.4% |
| γ1/K33 | 100.0% | 100.6% | 101.5% | 101.5% |

Tni is a phase transition temperature (e.g., a point of change from a nematic phase to an isotropic phase). When Tni is 80 degrees, a liquid crystal phase may be maintained up to 80 degrees, and the order of position of the liquid crystal may degrade at a temperature higher than 80 degrees, and there might not be a liquid crystal phase.

Δn represents refractive anisotropy of liquid crystal, and Δ∈ represents dielectric anisotropy of liquid crystal.

γ1 is a physical characteristic representing a rotational viscosity. It may represent on-off response characteristics, and the lower the yl, the faster the response may be.

K represents a modulus of elasticity of liquid crystals. There may be three types of modulus of elasticity: K11 (splay), K22 (twist), and K33 (bend). Splay may be exemplified as a characteristic of liquid crystals being spread out like a fan and then returning back to their original orientation. Twist may be exemplified as a characteristic where liquid crystals rotate along a horizontal axis by the application of an electric field and then return to their original orientation in a Polarized Light Source (PLS) mode. Bend may be exemplified as a force whereby liquid crystals are bent and then return to their original orientation in a Vertical Alignment (VA) mode. In a negative liquid crystal mode, when K33 is relatively large, the negative liquid crystals may return relatively quickly to their original orientation. This may mean that the lower the γ1/K33 is (e.g., the lower the γ1 (viscosity)), the higher the response speed may be, and the higher the K33 is, the higher the response speed may be.

The above table shows values of each characteristic relative to the values of the reference example.

As illustrated in table 3, as the amount of the 4-ring compound added increases, there may be a decrease in Δ∈ and an increase in γ1.

### Example 3

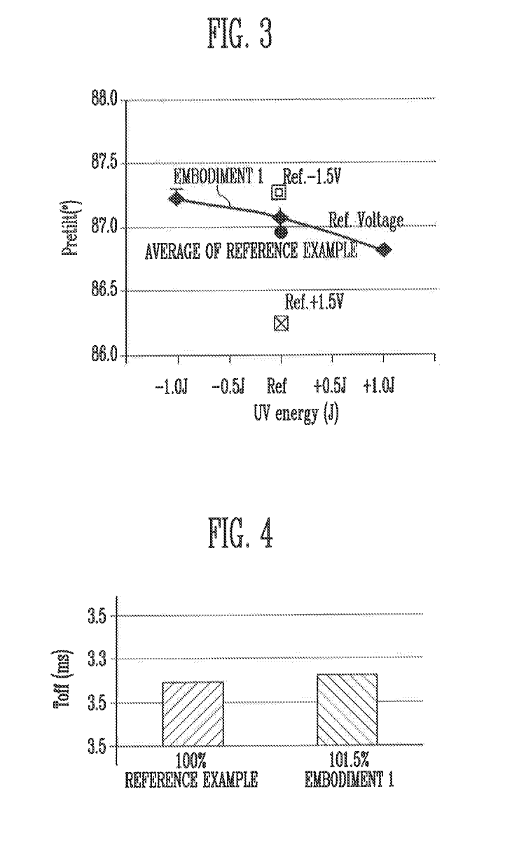

Optical characteristics of the liquid crystal composition of embodiment 1 and the reference example are shown in table 4 to table 6 and FIGS. 2 to 4.

Table 4 and FIG. 2 show measurements of threshold voltage Vth (V) of the liquid crystal composition including 0.3 wt % of the 4-ring compound according to UV energy (J). The Vth of the liquid crystal composition according to exemplary embodiments of the present invention may be similar to that of the reference example.

TABLE 4

| | Refer- | | | | | |
|---|---|---|---|---|---|---|
| | ence | | | | | |
| | example | Embodiment 1 | | | | |
| | Ref. | Ref. −1 V | Ref. −1 J | Ref. | Ref. +1 J | Ref. +1 V |
| Number | 4 | 4 | 4 | 4 | 4 | 4 |
| of cells | | | | | | |
| Min | 2.94 | 2.96 | 2.94 | 2.97 | 2.94 | 2.92 |
| Max | 2.97 | 3.00 | 2.99 | 2.98 | 2.95 | 2.95 |
| AVG | 2.96 | 2.98 | 2.97 | 2.97 | 2.95 | 2.93 |
| Stdev | 0.01 | 0.02 | 0.03 | 0.0 [symbol] | 0.01 | 0.01 |

[symbol]

indicates data missing or illegible when filed

Table 5 and FIG. 3 show pretilt angles (°) of the liquid crystal composition including 0.3 wt % of the 4-ring compound according to UV energy (J). The pretilt angle of the liquid crystal composition according to exemplary embodiments of the present invention may be similar to those of the reference example.

TABLE 5

| | Refer- | | | | | |
|---|---|---|---|---|---|---|
| | ence | | | | | |
| | example | Embodiment 1 | | | | |
| | Ref. | Ref. −1 V | Ref. −1 J | Ref. | Ref. +1 J | Ref. +1 V |
| Number | 4 | 4 | 4 | 4 | 4 | 4 |
| of cells | | | | | | |
| Min | 86.87 | 87.15 | 87.20 | 86.96 | 86.79 | 86.25 |
| Max | 87.07 | 87.38 | 87.25 | 87.11 | 86.84 | 86.46 |
| AVG | 86.96 | 87.26 | 87.22 | 87.07 | 86.82 | 86.35 |
| Stdev | 0.09 | 0.10 | 0.02 | 0.12 | 0.02 | 0.09 |

Table 6 and FIG. 4 illustrate response characteristic Toff (ms) of the liquid crystal composition including 0.3 wt % of the 4-ring compound. The response characteristics of the liquid crystal composition according to exemplary embodiments of the present invention may be similar to those of the reference example.

TABLE 6

| LC | Reference example | Embodiment1 |
|---|---|---|
| γ1 | 100% | 101% |
| γ1/K33 | 100% | 101.5% |
| Number of Cells | 4 | 4 |
| Min | 3.19 | 3.22 |
| Max | 3.19 | 3.23 |
| AVG | 3.19 | 3.23 |
| Stdev | 0.00 | 0.00 |

As illustrated in table 4 to table 6 and FIGS. 2 to 4, Vth, pretilt angle, and response characteristics of the liquid crystal composition including 0.3 wt % of the 4-ring compound may be similar to those of the liquid crystal of the reference example.

While the Invention has been particularly shown and described with reference to exemplary embodiments thereof, it will be understood by those of ordinary skill in the art that various changes in form and detail may be made therein without departing from the spirit and scope of the present invention.

What is claimed is:

1. A liquid crystal composition comprising an additive of chemical formula 1: 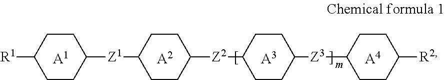
wherein R1 and R2 are each independently an alkyl having 1 to 12 carbon atoms, a hydrogen, a halogen or a cyano, respectively, and one or two nonadjacent CH₂ groups may be replaced by —O—, —CH═CH—, —CO—, —OCO— or —COO—, wherein an oxygen atom is not directly connected to another oxygen atom,
wherein A¹, A², A³, and A⁴ are each independently: 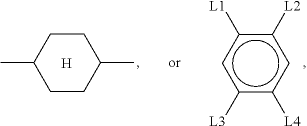
wherein one, two, or three radicals of A¹, A², A³ and A⁴ are each independently: 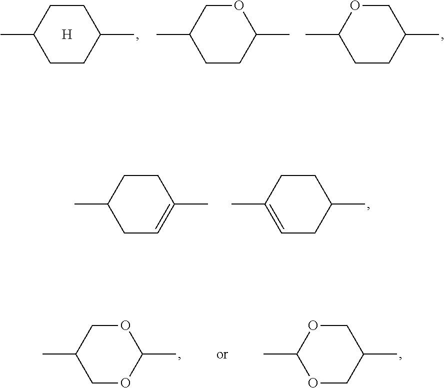
wherein L1 to L4 are each independently —H, —F, —Cl, —OCF₃, —CF₃, —CH₂F, or —CHF₂, and
wherein Z¹, Z² and Z³ are each independently —COO—, —OCO—, —CF₂O—, —OCF₂—, —CH₂O, —OCH₂—, —SCH₂—, —CH₂S—, —CH₂CH₂—, —C₂F₄—, —CH₂—CF₂—, —CF₂CH₂—, —(CH₂)z-, —CH═CH—, —CF═CF—, —CH═CF—, —CF═CH—, —C═C—, —CH═CHCH₂O—, or a single bond, and wherein when m=O, Z¹ and Z² do not represent a single bond at the same time, z is 3, 4, 5, or 6, and m is 0 or 1.

2. The liquid crystal composition according to claim 1,
wherein the additive of chemical formula 1 in the composition is 0.001 wt % to 0.35 wt % based on a total weight of the composition.

3. The liquid crystal composition according to claim 1,
wherein the liquid crystal composition comprises an alkenyl compound, an alkoxy compound, a hydroquinone compound, a terphenyl compound, or a tolane compound.

4. The liquid crystal composition according to claim 3,
wherein the alkenyl compound is at least one selected from: 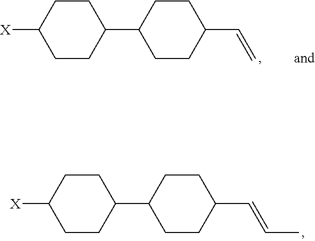
and wherein each X is an alkyl group comprising 1 to 5 carbon atoms.

5. The liquid crystal composition according to claim 3,
wherein the alkoxy compound is at least one selected from: 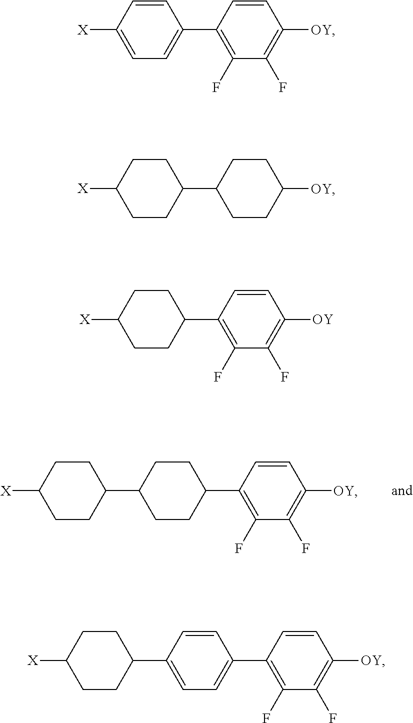
and wherein each of X and Y are an alkyl group comprising 1 to 5 carbon atoms.

6. The liquid crystal composition according to claim 3,
wherein the terphenyl compound is: 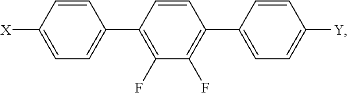
wherein each of X and Y are an alkyl group having 1 to 5 carbon atoms.

7. The liquid crystal composition according to claim 1,
wherein the liquid crystal composition does not comprise reactive mesogen (RM).

8. The liquid crystal composition according to claim 1,
wherein the liquid crystal composition comprises one or more types of reactive mesogen (RM).

9. The liquid crystal composition according to claim 1,
wherein the liquid crystal composition comprises a BHT or HALS type stabilizer.

10. The liquid crystal composition according to claim 9,
wherein the stabilizer comprises at least one compound selected from alkylate monophenol, alkylthiomethylphenol, hydroquinone and alkylate hydroquinone, tocopherol, hydroxylate thiodiphenyl ether, alkylidenebisphenol, O-, N- and S-benzyl compound, hydroxybenzylate malonate, aromatic hydroxybenzyl compound, benzylphosphonate, acylaminophenol, monovalent or multivalent alcohol, monovalent or multivalent alcohol and p-(3,5-di-tertbutyl-4-hydroxyphenyl)-propionic acid ester, monovalent or multivalent alcohol and p-(5-tertbutyl-4-hydroxy-3-methylphenyl)propionic acid ester, monovalent or multivalent alcohol and β-(3,5-dicyclohexyl-4-hydroxyphenyl)-propionic acid ester, monovalent or multivalent alcohol and 3,5-di-tertbutyl-4-hydroxyphenyl acetic acid ester, 1-(3,5-di-tertbutyl-4-hydroxyphenyl)propionic acid amide, ascorbic acid, and amine antioxidant, and a hindered amine compound.

11. The liquid crystal composition according to claim 1,
wherein the liquid crystal composition further comprises an optical initiator.

12. The liquid crystal composition according to claim 1,
wherein the liquid crystal composition is used in a liquid crystal device comprising a general VA orientation film, a PLS orientation film, or an orientation film comprising a reactive mesogen (RM), or no orientation film.
